(19) United States
(12) Patent Application Publication
(10) Pub. No.: US 2022/0168595 A1
(43) Pub. Date: Jun. 2, 2022

(54) TEETH CLEANING AND REMINERALIZING TOOTHPASTES

(71) Applicant: Robert Wayne Gustafson, Sun City West, AZ (US)

(72) Inventor: Robert Wayne Gustafson, Sun City West, AZ (US)

(21) Appl. No.: 17/404,754

(22) Filed: Aug. 17, 2021

Related U.S. Application Data
(60) Provisional application No. 63/120,246, filed on Dec. 2, 2020.

(51) Int. Cl. A61Q 11/00; A61K 8/29; A61K 8/60; A61K 8/34; A61K 8/46; A61K 8/73; A61K 8/21; A46B 9/04

(52) U.S. Cl. CPC A61Q 11/00; A61K 8/29; A61K 8/60; A61K 8/345; A61K 8/463; A61K 8/731; A46B 2200/1066; A46B 9/04; A61K 2800/413; A61K 2800/651; A61K 2800/87; A61K 2800/88; A61K 8/21

(57) ABSTRACT

A teeth cleaning and remineralizing toothpaste that includes food grade titanium dioxide (TiO₂) having a molecular size of 10 nanometers or less that is adapted to clean, polish, whiten, remineralize, and create a germ and stain barrier upon teeth used thereon over a prescribed period of time.

### CROSS-REFERENCE TO RELATED APPLICATION

The present application is related to and claims priority from prior provisional application Ser. No. 63/120,246, filed Dec. 2, 2020 which is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

### 1. Field of the Invention

The present invention relates generally to the field of tooth care and the cleaning and remineralizing of teeth prior to dental procedures by the use of a natural mineral within a toothpaste.

### 2. Description of the Related Art

Toothpastes have been used in the past to clean and whiten teeth. However, none of the prior art toothpastes have been able to reach deep enough into cracks and cavities to eliminate all the germs that caused them nor to remineralize the teeth to help repair them or prepare them sufficiently for needed dental procedures. As a result, decay would continue, and when dental work was to be performed a dentist would have to remove more of the effected teeth to remove all the germs present before repairing the teeth properly.

Therefore, a need exists for toothpaste that includes a natural mineral sized to go deeper into existing cavities than the germs themselves, clean them out, and then fill them in via remineralization to avoid the above-mentioned problems.

### BRIEF SUMMARY OF THE INVENTION

In view of the foregoing disadvantages inherent in known toothpastes a teeth cleaning and remineralizing toothpaste is set forth herein that includes 10 nanometers or below of food grade Titanium Dioxide (TiO₂) and is adapted to clean, polish, whiten, remineralize, and create a germ and stain barrier upon teeth used thereon over a prescribed period of time.

The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate the effect of the teeth cleaning and remineralizing toothpaste set forth herein according to the teachings of the present invention.

FIG. 1 shows a tooth 100 before the use of the instant teeth cleaning and remineralizing toothpaste of the instant invention.

FIG. 2 shows the tooth 100 after the use of the instant teeth cleaning and remineralizing toothpaste of the instant invention used twice a day for four weeks.

FIG. 3 shows a tooth 300 before the use of the instant teeth cleaning and remineralizing toothpaste of the instant invention.

FIG. 4 shows the tooth 300 after the use of the instant teeth cleaning and remineralizing toothpaste of the instant invention used twice a day for four weeks.

The various embodiments of the present invention will hereinafter be described in conjunction with the appended drawings.

### DETAILED DESCRIPTION

As discussed above, the present invention relates to a teeth cleaning and remineralizing toothpaste includes up to 10 nanometers of food grade Titanium Dioxide (TiO₂) and is adapted to clean, polish, whiten, remineralize, and create a germ and stain barrier upon teeth used thereon over a prescribed period of time.

The size of most cavity causing germs are 3 micrometers (3,000 nanometers). The size of the Titanium Dioxide (TiO₂) used in the instant toothpaste is 10 nanometers or less. The size difference allows the TiO₂ to go deeper into cavities than the germs, clean them out, and then fill in the cracks and microscopic holes caused by the germs via remineralization between the material the teeth are formed from and the Titanium Dioxide (TiO₂) used. As such, a layer of remineralized tooth is created by the chemical reaction and covers the outer layer of the tooth including the area that has been damaged, ie., the cracks and microscopic holes of a cavity.

In use, an individual would use the teeth cleaning and remineralizing toothpaste prior to a dental procedure for a prescribed period of time to thereby clean the teeth and remineralize the teeth or tooth, thereby reducing the need for a dentist to remove larger portions of each tooth to make sure that all the germs are removed. The instant invention reduces the need to remove larger portions of the teeth, reduces the pain caused by the more intrusive dental procedures, and reduces the cost to the patient.

The recommended daily use of the instant teeth cleaning and remineralizing toothpaste is usually no more than four weeks. Further use is only advised if under instructions by a dental professional.

By eliminating the germs and remineralizing the tooth to an extent gives a dentist a more targeted area to drill/grin, thereby less tooth to remove. In some cases, just a light cleaning and smaller filling is necessary.

All the ingredients used within the instant teeth cleaning and remineralizing toothpaste are on the FDA and ADA approved ingredients list. The ingredients may include water; sorbitol; glycerin; sodium lauryl sulfate; cellulose gum; fluoride; abrasives; hydrated silica; poloxamer 407; aroma; PEG-12; sodium saccharin; and tri-calcium phosphate. Other ingredients may also be included for other purposes.

The percentage by volume of the titanium dioxide within the instant teeth cleaning and remineralizing toothpaste in a preferred embodiment is 2% or less.

The desired name and/or best descriptive word for the instant teeth cleaning and remineralizing toothpaste is “Nanopaste”.

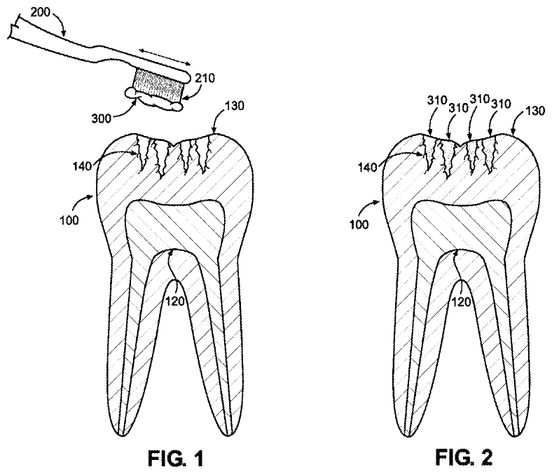

Referring now to FIGS. 1 and 2, a tooth 100 is shown before and after the use of the instant teeth cleaning and remineralizing toothpaste used twice a day for four weeks. FIG. 1 shows tooth 100 with cavities or cracks 140, the pulp section of the tooth 120, and the top portion of the tooth 130. Further, toothbrush 200 is shown with bristles 210. The teeth cleaning and remineralizing toothpaste 300 is shown upon the bristles 210 for use in brushing the tooth 100. FIG. 2 shows the teeth cleaning and remineralizing toothpaste 300 within the cavities or cracks 140.

In use, the continuous brushing of the teeth will break off the weaker parts of a tooth. The titanium oxide kills bacteria and germs and eventually remineralizes the tooth and helps fill in a portion of any cracks, dents, and cavities. Many of the cracks, dents, and cavities of up to 5 nanometers can be filled in by this toothpaste and process. And bacteria as small as 3 microns are killed. And, at around four weeks of use the tooth is ready for a dentist to finish filling in any of the cracks, dents, and cavities much more easily and completely.

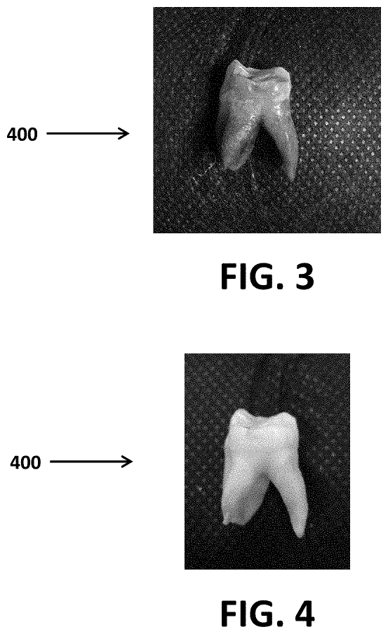

Referring now to FIGS. 3 and 4, a tooth 400 is shown before and after the use of the instant teeth cleaning and remineralizing toothpaste used twice a day for four weeks. It is shown that the teeth cleaning and remineralizing toothpaste 300 is effective in removing stains that are upon and below the outer layers of the tooth 400.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is:

1. In a paste for cleaning teeth, the improvement comprising:
titanium dioxide;
wherein the molecular size of the titanium dioxide is 10 nanometers or less;
wherein said paste is adapted to clean, polish, whiten, remineralize, and create a germ and stain barrier upon teeth used thereon over a prescribed period of time.

2. The paste for cleaning teeth of claim 1, wherein the proportion by volume of said titanium dioxide within said paste is 2% or less.

3. The paste for cleaning teeth of claim 1, wherein said molecular size of the titanium dioxide is 10 nanometers.

4. The paste for cleaning teeth of claim 1, wherein the proportion by volume of said titanium dioxide within said paste is 2%.

5. A toothpaste for cleaning and remineralizing teeth, comprising:
water;
sorbitol;
glycerin;
sodium lauryl sulfate;
cellulose gum;
fluoride;
abrasives; and
titanium dioxide;
wherein the molecular size of the titanium dioxide is 10 nanometers or less;
wherein said toothpaste is adapted to clean, polish, whiten, remineralize, and create a germ and stain barrier upon teeth used thereon over a prescribed period of time.

6. The toothpaste for cleaning teeth of claim 5, wherein the proportion by volume of said titanium dioxide within said paste is 2% or less.

7. The toothpaste for cleaning teeth of claim 5, further comprising:
hydrated silica;
poloxamer 407;
aroma;
PEG-12;
sodium Saccharin; and
tri-calcium phosphate.

8. The toothpaste for cleaning teeth of claim 5, wherein said molecular size of the titanium dioxide is 10 nanometers.

9. The toothpaste for cleaning teeth of claim 5, wherein the proportion by volume of said titanium dioxide within said paste is 2%.

10. A tooth cleaning kit comprising:
a toothbrush including:
bristles; and
a toothpaste for cleaning and remineralizing teeth, comprising:
water;
sorbitol;
glycerin;
sodium lauryl sulfate;
cellulose gum;
fluoride;
abrasives; and
titanium dioxide;
wherein the molecular size of the titanium dioxide is 10 nanometers or less;
wherein said toothpaste is adapted to be placed upon said bristles of said toothbrush and used to clean, polish, whiten, remineralize, and create a germ and stain barrier upon teeth used thereon over a prescribed period of time.

11. The kit of claim 10, wherein the proportion by volume of said titanium dioxide within said paste is 2% or less.

12. The kit of claim 10, further comprising:
hydrated silica;
poloxamer 407;
aroma;
PEG-12;
sodium Saccharin; and
tri-calcium phosphate.

13. The kit of claim 10, wherein said molecular size of the titanium dioxide is 10 nanometers.

14. The kit of claim 10, wherein the proportion by volume of said titanium dioxide within said paste is 2%.
